(19) United States
(12) Patent Application Publication
(10) Pub. No.: US 2025/0281325 A1
(43) Pub. Date: Sep. 11, 2025

(54) TEMPERATURE CONTROL DEVICE FOR FACE PADS

(71) Applicant: AUROX GMBH, Graz (AT)

(72) Inventor: Christoph Schoeggler, Graz (AT)

(21) Appl. No.: 18/862,718

(22) PCT Filed: May 4, 2023
(86) PCT No.: PCT/EP2023/061812

§ 371 Date: Nov. 4, 2024

(30) Foreign Application Priority Data
May 4, 2022 (AT) A50306/2022

(51) Int. Cl. A61F 7/00

(52) U.S. Cl. CPC A61F 7/007; A61F 2007/0004; A61F 2007/0075; A61F 2007/0077; A61F 2007/0086; A61F 2007/0095

(57) ABSTRACT

The present invention relates to a temperature control device for face pads, comprising: a Peltier element having a receiving region for receiving a face pad, the receiving region corresponding to a warm side or a cold side of the Peltier element; and a retaining magnet apparatus for magnetically fastening a face pad in the receiving region, the retaining magnet apparatus being disposed, in relation to the Peltier element, on the side facing away from the receiving region.

### CROSS-REFERENCE TO RELATED APPLICATIONS

This application is a U.S. National Stage Application under 35 U.S.C. § 371 of International Application No. PCT/EP2023/061812, filed May 4, 2023, titled TEMPERATURE CONTROL DEVICE FOR FACE PADS, which claims priority to Austrian Application No. 50306/2022, filed May 4, 2022. International Application No. PCT/EP2023/061812 is incorporated by reference herein in its entirety.

### TECHNICAL FIELD

The invention relates to temperature control devices for support elements, being temporarily applied to the facial and in particular to the periocular area, such as the so-called eye pads or philtrum pads.

### BACKGROUND

Support elements that can be applied cutaneously and removed again in the periocular facial area, also known as “eye pads”, are products used in the cosmetics industry to be applied by a user in the area under and/or around the eyes so as to achieve a cosmetic effect on the skin of the user. For reducing the formation of wrinkles in the cutaneous area between the nose, nasolabial folds and upper lip, known as the philtrum, it is possible to use support elements that can be applied cutaneously, also known as “philtrum pads”. In particular, the targeted cutaneous application of heat or cold can be an effective means of treating pleated wrinkles.

In the prior art, for example, there are known, for example, eye pads containing an active ingredient that is slowly released onto the user's skin. This can, for example, stimulate blood circulation or, in the case of moisturizers, reduce the evaporation of water from the skin's surface, thereby improving moisture retention in the skin.

Furthermore, therapies for the treatment of eye diseases such as meibomian gland dysfunction (MGD)—a chronic abnormality of the meibomian glands (MG)—or other unpleasant or pathological changes in and around the eye can be positively supported by targeted heating or cooling of the periocular area.

In the prior art there are known devices that heat and/or cool the skin under the user's eyes. It is, therefore an ordinary practice to use natural agents such as cucumber slices or similar, which have a cooling effect by dissipating evaporation heat. There are however also known technical devices that are applied to the skin and cause the skin to be heated or cooled by means of electrical, chemical or biochemical components.

### SUMMARY

One of the ideas of the present invention is to provide a temperature control device for a defined heating or cooling of eye pads or philtrum pads.

A first aspect of the invention relates to an eye pad comprising an eye pad body having a first side for contacting a skin of a user, and a second side substantially opposite the first side. The eye pad body comprises a passage from the first side to the second side, and the eye pad comprises a negative pressure generating element disposed on the second side at one end of the passage, and preferably extending at least partially into the passage. According to the invention, the negative pressure generating element is at least partially elastic and is configured to provide a suction force in the passage through the eye pad body as a result of elastic deformation of the negative pressure generating element when the first side of the eye pad body is placed on the skin of the user.

By designing the eye pad according to the first aspect of the invention with the passage from the first side to the second side through the eye pad body and the negative pressure generating element, a secure fastening method is provided which does not require additional mechanical retaining means. This ensures a secure fastening of the eye pad under one of the user's eyes, which is easy to use and does not cause any unpleasant pressure points in the user's head area, resulting in a high level of wearing comfort. The suction effect also improves blood circulation in the area where the eye pad is attached. The eye pad of the first aspect of the invention prevents uncontrolled falling off from the skin of the user, without severely restricting the user's freedom of movement while using the eye pad. The eye pads are also easy to use and do not require temperature control devices to be coupled during use.

According to some embodiments of the eye pad according to the invention, the passage comprises a larger opening on the first side than on the second side. This increases the area on the user's skin to which the suction force is distributed. This reduces the likelihood of the suction effect damaging vessels in the user's skin. In addition, this further improves the wearing comfort of the eye pads according to the invention.

Preferably, the negative pressure generating element may comprise an extension protruding from the second side. This makes it easier for the user of the eye pad according to the invention to grip the negative pressure generating element.

According to some embodiments, the passage comprises a plurality of slots and/or holes substantially oriented in a passage direction. This divides the passage into several sections, wherein the suction effect is distributed over the several slots and/or holes. This distributes the force generated by the suction effect over a larger area of the skin surface.

According to some embodiments of the eye pad according to the invention, the slots and/or holes are disposed at an acute angle to each other, narrowing in the direction of the second side. This further increases the area of the user's skin that is exposed to the suction effect.

According to some alternative embodiments of the eye pad according to the invention, the negative pressure generating element extends from the second side to the first side of the eye pad body through the passage, and is designed as an at least partially elastic insert in the eye pad body which is open on one side in the direction of the first side. This results in an improved seal between the negative pressure generating element and the eye pad body, which means that the negative pressure and the resulting suction effect can be maintained over a longer period of time.

Preferably, the negative pressure generating element may comprise a flexible lip arranged on the first side of the eye pad body and surrounding the passage. This provides an improved seal between the passage through the eye pad body and the user's skin. In addition, the passage may comprise, for example, a narrow point between the first side and the second side of the eye pad body. As a result, the extent to which the user's skin can be sucked into the eye pad body is limited.

According to some variant embodiments of the eye pad according to the invention, the eye pad body is made of a metal, a plastic, a ceramic or combinations thereof, and can advantageously be coated with a soft material such as silicone, latex and/or rubber. By manufacturing the eye pad body from metal, an increased heat capacity of the eye pad body is achieved, which means that the warming or cooling effect on the user's skin can be maintained over a longer period of time when the eye pad is heated or cooled. Covering the eye pad body with silicone, latex and/or rubber provides a soft surface that is pleasant to the touch and also thermally insulates the eye pad body. As a result, the risk of burns or frostbite occurring on the user's skin when using the eye pad is reduced.

According to a second aspect of the invention, a temperature control device for eye pads and/or philtrum pads comprises a Peltier element having a receiving region for receiving a face pad, wherein the receiving region corresponds to a warm side or a cold side of the Peltier element. Further, the temperature control device contains a retaining magnet apparatus for magnetically fastening a face pad in the receiving region, wherein the retaining magnet apparatus is disposed in relation to the Peltier element on the side facing away from the region receiving it.

This means that an eye pad or philtrum pad, which comprises a magnetizable material or also a magnet, can be firmly fixed in the receiving region. It also follows that the contact between the eye pad or philtrum pad and the Peltier element corresponds to a predetermined quality, for example in compliance with limit values of quality parameters such as flatness, surface roughness or the like. In this respect, the eye pad or philtrum pad, for example, has the largest possible contact surface to the Peltier element, i.e. the contact surfaces between the Peltier element and the eye pad have a complementary outer shape.

According to some embodiments of the temperature control device, the Peltier element, at least in the receiving region, comprises a coating for mechanical protection of the Peltier element surface. The coating within the meaning of this application is created by applying a firmly adhering layer of shapeless material to the surface of a workpiece. The coating can be either individual layers or several contiguous layers. In this respect, the coating can be applied using a chemical, mechanical, thermal and/or thermomechanical coating process. The coating has, for example, a substrate which is configured as a liquid, in particular as a paint, or as a solid, in particular as a powder, laminate film or comparable film, before the coating process.

In this way, mechanical damage such as scratches or paint removal on the Peltier element can be avoided.

According to a further development of the temperature control device, the coating has a thickness of at most 200 μm, preferably at most 100 μm. This means that the heat transfer from the Peltier element to the eye pad can only be influenced insignificantly. Alternatively, or additionally, the coating has a thermally conductive material.

According to some embodiments, the temperature control device further comprises a chargeable energy store which is electrically connected to the Peltier element. In this way, the temperature control device is transportable, which is therefore advantageous for traveling in particular, and can therefore be used independently of a stationary energy source.

According to some embodiments of the temperature control device, the retaining magnet apparatus has at least two magnets or parts made of magnetizable material for magnetically fastening a face pad. As a result, the orientation and/or position of the pad in the receiving region can be predetermined so that, for example, the receiving region is used as spatially as possible or the pads are attached at points with defined heat transfer.

According to some embodiments, the temperature control device further comprises a fixing device which fixes the Peltier element on the side of the receiving region. The fixing device is produced, for example, from a glass fiber reinforced plastic. It can surround the receiving region in portions and be fastened to a base of the temperature control device. In this respect, the fixing device can be fastened to the base by a detachable fastening means, in particular by a screw connection, a plug-in connection or the like. In this way, the Peltier element can be pressed against the base under a predetermined contact pressure to create a defined contact.

According to some embodiments of the temperature control device, a temperature sensor is disposed in the receiving region on a surface of the Peltier element and a control unit is connected to the temperature sensor and the Peltier element. The control unit is configured to regulate the heating or cooling power of the Peltier element on the basis of the temperature of the Peltier element surface in the receiving region detected by the temperature sensor. In this respect, more than one temperature sensor can also be provided at different measuring positions within the temperature control device. Further, other sensors such as voltage sensors or current sensors can also be implemented at suitable points in the temperature control device.

According to a further development of the temperature control device, the control unit is further configured to regulate the heating or cooling power of the Peltier element when a target temperature of the Peltier element surface in the receiving region is reached, such that the temperature of the Peltier element surface in the receiving region remains substantially constant. In this respect, the Peltier element can be alternately supplied with or without electrical voltage so that current flows temporarily through the Peltier element. For example, the control unit switches a circuit in which the Peltier element and a power source are integrated on or off depending on the detected temperature compared to the target temperature.

According to a further development of the temperature control device, the control unit is further configured to take into account an ambient temperature measured by an ambient temperature sensor in the controlled heating or cooling power in such a way that the heating or cooling power is increased, reduced or prevented in predetermined temperature ranges of the ambient temperature. For example, in an environment whose temperature is higher than the target temperature, further heating of the eye pad or philtrum pad can be prevented. Optionally, in an environment with an ambient temperature below a usual room temperature, the heating power can be increased to compensate for the heat lost from the eye pads or philtrum pads to the environment.

According to a further development of the temperature control device, if the temperature control device has at least two Peltier elements being in operation simultaneously, the control unit is further configured to supply the current from a current source in phases to only one of the at least two Peltier elements. In a temperature control device with two Peltier elements, one of the two Peltier elements can thus be heated or cooled more quickly. The power source is, for example, the chargeable energy store or the power grid, provided the temperature control device is connected to the power grid via a power cable.

According to a further embodiment, the temperature control device also comprises a base that supports all components of the temperature control device. The base is preferably made of a temperature conducting material. This improves heat dissipation from the side of the Peltier element facing away from the receiving region to the environment when the receiving region corresponds to the cold side of the Peltier element, so that the heat not required on the warm side is not accumulated in the temperature control device. The thermal coupling between the base and the side facing away from the receiving region of the Peltier element can be provided by surface contact, for example. In addition, a thermally conductive paste, a preferably plastically deformable thermally conductive pad or a comparable thermally conductive material can fill any cavities in the surface contact and thus support the heat transfer between the Peltier element and the base.

According to a third aspect of the invention, a pad kit comprises at least one, preferably at least two eye pads, and/or a philtrum pad, and a temperature control device. The temperature control device has a Peltier element and a receiving region for at least two eye pads. The eye pads are thus thermally coupled to the Peltier element by direct contact with a warm side or a cold side of the Peltier element. The temperature control device makes it possible to heat or cool one or more face pads according to the invention.

According to one embodiment, the pad kit comprises two eye pads according to the invention and a common receiving region for receiving the two eye pads.

According to the preferred embodiment of the pad kit according to the invention, the temperature control device comprises at least one cover spanning the receiving region. As a result, the face pads are protected against mechanical damage. This cover also preferably includes a hinge, which makes it easy to open and close the cover.

According to the preferred embodiment of the pad kit according to the invention, the Peltier element surface is coupled to a temperature sensor, and the temperature control device comprises a control unit connected to the temperature sensor and the Peltier element, which is configured to regulate a heating or cooling power of the Peltier element on the basis of a temperature of the Peltier element surface detected by the temperature sensor. This prevents the face pads from being heated to a temperature or cooled to a temperature that would supply burn or frostbite symptoms when applied to a user's skin.

Preferably, the control unit is configured to regulate the heating or cooling power of the Peltier element when the target temperature of the Peltier element surface is reached, so that the temperature of the Peltier element surface is kept substantially constant. This keeps the face pads at a constant temperature that is optimal for an application.

### BRIEF DESCRIPTION OF THE DRAWINGS

Preferred and alternative embodiments of the eye pad according to the invention and the pad kit according to the invention are explained in more detail below on the basis of the figures.

FIG. 1 shows two face pads according to the invention during application.

FIG. 2a to FIG. 8b show different embodiments of the face pads according to the invention in a cross-sectional view.

FIGS. 9a and 9b show a pad kit according to the invention with two eye pads and a temperature control device according to the invention, with FIG. 9b showing an exploded view of the pad kit.

### DETAILED DESCRIPTION OF SOME EMBODIMENTS

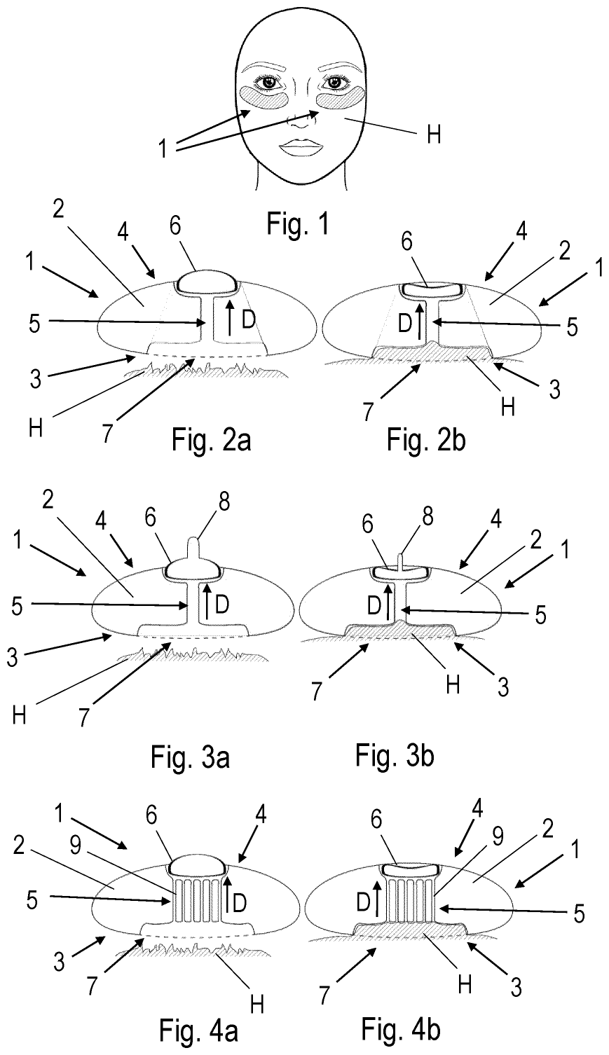

FIG. 1 shows two face pads 1 according to the invention, which have been placed on a user's skin H under the eyes. The face pads can thus be referred to as eye pads 1 and are used for cosmetic treatment of the area under the user's eyes covered by the eye pads 1.

In the course of such an exemplary treatment, the eye pads 1 according to the invention are first heated to a first target temperature, such as 40° C., then applied to the user's skin H under the eyes or in the periocular area, and fastened by means of a suction force. After the skin H has been warmed by the eye pads 1, they are removed again and a cream, preferably an eye cream such as an anti-wrinkle cream, is applied to the periocular area of the user's eyes in the area warmed by the eye pads. Subsequently, the same or additional eye pads, which have been cooled to a second target temperature, such as between 5° C. and 12° C., are applied to these areas and fastened again using suction force. The heating effect favors the receptacle of the cream by the skin H, wherein the subsequent cooling helps to close the pores of the skin H again. This improves the effect of the applied skin cream.

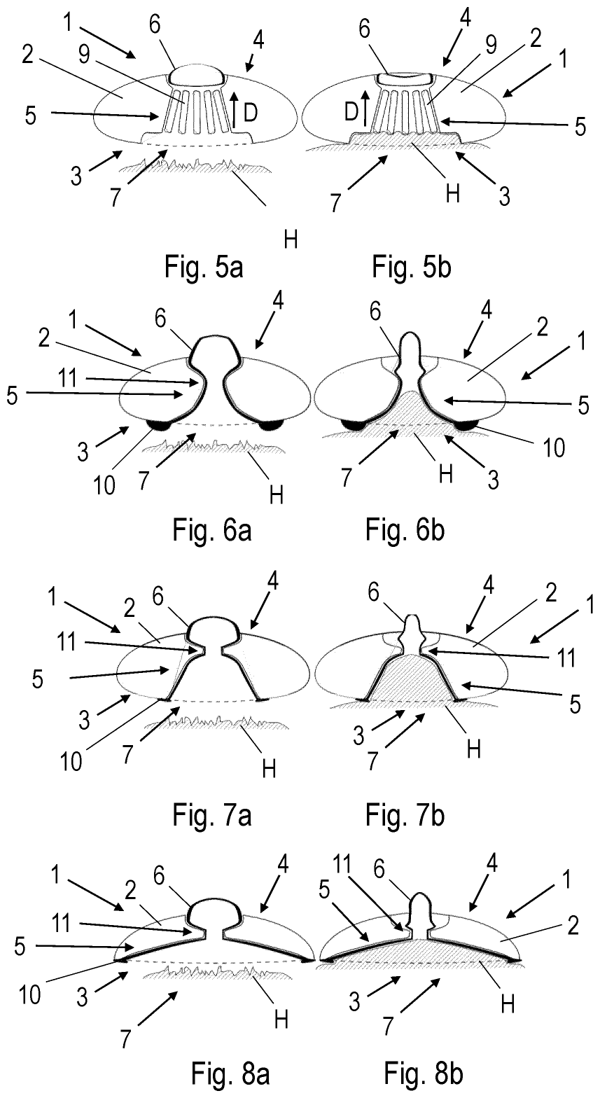

A sectional view of an eye pad 1 according to the invention is shown in FIG. 2a. The eye pad 1 according to the invention comprises an eye pad body 2, a first side 3 for resting on the user's skin H, and a second side 4 substantially opposite the first side 3. The eye pad body 2 comprises a passage 5 from the first side 3 to the second side 4. The eye pad 1 also comprises a negative pressure generating element 6 which is disposed on the second side 4 at one end of the passage 5 and preferably extends at least partially into the passage 5. The negative pressure generating element 6 is at least partially elastic and can, for example, be designed as a hollow body with an at least partially elastic shell comprising a hole oriented in the direction of the passage 5. The negative pressure generating element 6 is configured to provide a suction force in the passage 5 through the eye pad body 2 as a result of an elastic deformation of the negative pressure generating element 6 when the first side 3 of the eye pad body 2 is placed on the skin H of the user. When attaching the eye pad 1 according to the invention, the first side 2 is thus placed on the user's skin H, and subsequently or simultaneously a force is exerted on the negative pressure generating element 6. This reversibly deforms the negative pressure generating element 6. Due to the reversible deformation, if the negative pressure generating element 6 is configured as an at least partially elastic hollow body as described above, a volume of the negative pressure generating element 6 is reduced. As the passage 5 on the first side 3 is blocked by the user's skin H, a suction effect is created in the passage 5. As a result, the eye pad 1 is sucked onto the user's skin H and reversibly fastened to it. This state is shown in FIG. 2b. In FIG. 2a to FIG. 8b, in the figures marked with the letter b, the respective embodiment of the eye pad 1 according to the invention is always shown in the state in which it is suctioned onto the user's skin H, i.e. in the attached state.

As can be seen in FIGS. 2a to 8b, the passage 5 preferably comprises a larger opening 7 on the first side 3 than on the second side 4. This increases the area on the user's skin H to which the suction force is distributed. This reduces the likelihood of the suction effect damaging vessels in the user's skin H. In addition, this further improves the wearing comfort of the eye pad 1 according to the invention.

FIG. 3a shows the eye pad 1 according to the invention in an alternative embodiment. According to this embodiment, the negative pressure generating element 6 comprises an extension 8 protruding from the second side 3. This makes it easier for the user of the eye pad 1 according to the invention to grip the negative pressure generating element 6 and deform it.

According to the preferred embodiment shown in FIG. 4a and FIG. 4b, the passage 5 comprises a plurality of slots and/or holes 9 oriented substantially in a passage direction D. This divides the passage 5 into several sections, whereby the suction effect is distributed over the several slots and/or holes 9. This distributes the force generated by the suction effect over a larger area of the surface of the skin H.

According to an embodiment variant of the preferred embodiment of the eye pad 1 according to the invention, as shown in FIG. 5a and FIG. 5b, the slots and/or holes 9 are disposed at an acute angle to one another, narrowing in the direction of the second side 4. This further increases the area of the user's skin H that is exposed to the suction effect.

FIG. 6a to FIG. 8b show embodiments of the eye pad 1 according to the invention, in which the negative pressure generating element 6 extends from the second side 4 to the first side 3 through the passage 5. The negative pressure generating element 6 is designed as an at least partially elastic insert in the eye pad body 2 that is open on one side in the direction of the first side 3. By designing the negative pressure generating element 6 as an insert in the eye pad body 2 that is open on one side, the advantage is achieved that a better seal is achieved between the negative pressure generating element 6 and the eye pad body 2, as well as the skin H of the user. Preferably, as shown in FIG. 6a and FIG. 6b, the negative pressure generating element 6 comprises a flexible lip 10 arranged on the first side 3 of the eye pad body 2 and surrounding the passage 5. The flexible lip 10 further improves the seal against the user's skin H, allowing the eye pad 1 to adhere better to the user's skin H.

As can be seen in FIG. 6a to FIG. 8b, the passage 5 may comprise a narrow point 11 between the first side 3 and the second side 4. As a result, the extent to which the user's skin H can be sucked into the eye pad body 2 is limited.

According to the preferred embodiment of the eye pad 1 according to the invention, the eye pad body 2 is made of a metal, and preferably covered with a soft material such as silicone, latex and/or rubber. This results in an increased heat capacity of the eye pad body 2, whereby the warming or cooling effect on the user's skin H can be maintained over a longer period of time when the eye pad is heated or cooled. Alternatively or additionally, the interior of the eye pad body 2 may thus comprise a temperature in the range of 55° C. to 65° C., while the outer surface of the eye pad 1, with or without coating, would comprise a temperature in the range of 35° C. to 45° C. Covering the eye pad body 2 with silicone, latex and/or rubber provides a soft surface that is pleasant to the touch and also thermally insulates the eye pad body 2. As a result, the risk of burns or frostbite occurring on the user's skin H when using the eye pad 1 is reduced.

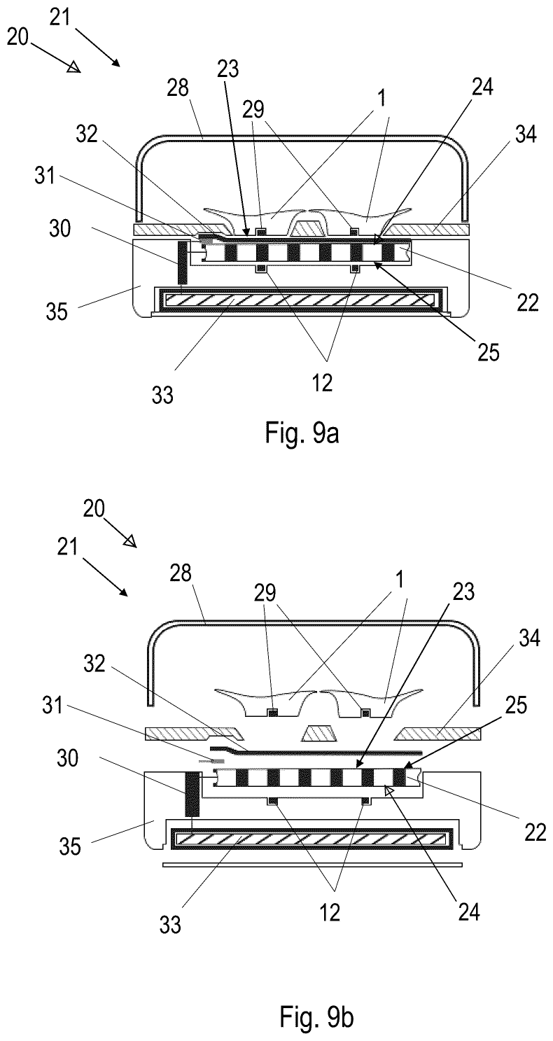

FIGS. 9a and 9b show a schematic representation of a pad kit 20 according to the invention, which comprises two face pads 1 according to the invention. These face pads 1 are shown in FIGS. 9a and 9b as eye pads 1, but it should be clear that the face pads 1 can also have other support elements, such as pads that can be applied to the cutaneous area between the nose, upper lip and nasolabial folds (“philtrum pads”). In the following description of exemplary embodiments, reference is made to eye pads—however, analogous technical considerations apply equally to other face pads 1 such as, in particular, philtrum pads.

The kit 20 further comprises a temperature control device 21 according to the invention, which has a Peltier element 22 with a receiving region 23 for receiving an eye pad 1. The receiving region 23 corresponds to either a warm side 25 or a cold side 24 of the Peltier element 22. This provides a means of heating or cooling the eye pads 1 before they are applied to the user's skin H.

In addition, the temperature control device 21 has a retaining magnet apparatus 12 for magnetically fastening a pad 1 in the receiving region 23. In this respect, the retaining magnet apparatus 12 is disposed in relation to the Peltier element 22 on the side facing away from the receiving region 23 24; 25, i.e. below the Peltier element 22. The magnetic retaining force of the retaining magnet apparatus 12 thus acts through the Peltier element 22. Here, the skilled person provides the retaining magnet apparatus 12 depending on the type of material of the surrounding components and/or the distance to the receiving region 23 in such a way that the magnetic retaining force in the receiving region 23 is strong enough to fix an eye pad 1 according to the invention.

Further, the Peltier element 22 exemplarily has a coating 32 for mechanical protection of the Peltier element surface. The coating 32 is as an example configured as a laminate film. In this respect, the coating 32 is applied to the upper side of the Peltier element 22, which side also has the receiving region 23. In addition, the coating 32 may also be applied to the lower side.

This coating 32 can also be provided in all design variants of the kit according to the invention 20.

Preferably, a temperature sensor 31 is further provided. The temperature sensor 31 is provided, for example, between the Peltier element 22 and the coating 32. In particular, the temperature sensor 31 is disposed at the edge of the receiving region 23. For example, the temperature sensor 31 is also arranged on the coating 32, arranged in a recess in the coating 32 or embedded in the Peltier element 22. To embed a temperature sensor 31 in the Peltier element 22, it may be possible to create recesses within the Peltier element into which a temperature sensor 31, for example based on an NTC element on a printed circuit board, is inserted and wired to the outside. The embedded temperature sensor 31 then touches, in the Peltier element 22, one side of the Peltier element 22, respectively, so that differential measurements can also be carried out. Optionally, a plurality of temperature sensors 31 may be provided, which are selectively provided in the aforementioned positions or alternative positions.

Optionally, the temperature control device 21 has a chargeable energy store 33. The energy store 33 is at least electrically connected to the Peltier element 22, but can also be electrically connected to other components of the temperature control device 21. The chargeable energy store shown in FIGS. 9a and 9b is, for example, an accumulator which is chargeable by cable or wirelessly, in particular inductively, resonantly or the like. Furthermore, the energy storage unit 33 can be integrated into the temperature control device 21 in a fixed or replaceable manner. Further, in some variants it may be possible to dispense with the energy storage unit 33 and instead provide a completely grid-connected power supply.

The receiving region 23 of the Peltier element 22 can also be used, for example, to cool an eye roller or the like as part of the kit 20 according to the invention. Additionally, or alternatively, the temperature control device 21 may also comprise a mounting means for the eye roller and/or a cream jar, wherein the eye roller and/or the cream jar, in a state received on the temperature control device 21, are thermally coupled to the cold side 25 or the warm side 24 of the Peltier element 22. This has the advantage that the eye roller and/or cream jar can be heated or cooled at the same time as the eye pads 1.

As shown in FIGS. 9a and 9b, the eye pads 1 according to their preferred embodiment may each comprise at least one magnet 29 or a magnetizable material for magnetically fastening the eye pads 1 in the receiving region 23. This provides a secure fastening of the eye pads 1 to the receptacles 23. Alternatively, the eye pads 1 can also have a magnetizable material.

In addition, the temperature control device 21 includes, for example, a base 35, which carries at least the Peltier element 22, the retaining magnet apparatus 12 and the energy store 33. In addition, the base 35 can have a temperature conducting material in order to receive the waste heat from the Peltier element 22 and release it to the environment. In this way, overheating of the temperature control device 21 can be reduced. In addition, the base 35 may have a temperature sensor that measures the temperature of the base. This means that the temperature of the base can be used as a reference value for the temperature of the Peltier element surface, for example to throttle the Peltier element if the temperature of the base is too high in order to prevent overheating.

According to an alternative variant embodiment of the kit 20 according to the invention, the receiving region 23 is delimited by a fixing device 34, which is thermally decoupled from the Peltier element 22. The fixing device 34 fastens the Peltier element 22 and, if applicable, the coating 32 to the base 35 by means of a screw connection.

According to one variant embodiment of the kit 20 according to the invention, the kit 20 comprises two eye pads 1 and two receiving regions 23, each receiving region 23 receiving one eye pad 1. Optionally, the fixing device 34 is configured as a trough substantially adapted to the shape of the eye pads 1. In addition, the temperature control device 21 preferably comprises at least one cover 28 spanning at least one receiving region 23. In addition, the covers 28 preferably each comprise or include a hinge, which is not evident in the figures. This provides a swivel connection of the cover 28 with the temperature control device 21. In addition, the cover 28 can include a magnet for reversible closing of the cover 28.

In an alternative variant embodiment of the eye pad kit 20 according to the invention, two Peltier elements 22 are provided, each having its own receiving region 23. In addition, a control unit 30 can be provided to control the respective Peltier elements 22. This control unit 30 can also be provided in variant embodiments of the temperature control device 21 with only one Peltier element 22, as shown in FIGS. 9a and 9b. Depending on the orientation or control of the Peltier elements 22 by the control unit 30, the receiving region 23 corresponds with the warm side 24 or the cold side 25 of the respective Peltier element 22. Preferably, each of the receiving regions 23 also has a temperature sensor 31. The control unit 30 is connected to the temperature sensors 31 and the Peltier element 22. The control unit 30 regulates the heating or cooling power of the Peltier element 22 or the Peltier elements 22 on the basis of the temperature of one or more of the Peltier element surfaces detected by the temperature sensors 31, for example in the receiving region 23 or—in the case of a temperature sensor 31 embedded in the Peltier element 22—on the inside top and bottom of the Peltier element 22. In addition, when a target temperature of the Peltier element surface in the receiving region 23 is reached, the control unit 30 can regulate the heating or cooling power of the Peltier element 22 in such a way that the temperature of the Peltier element surface in the receiving region 23 is kept substantially constant. For example, it is also possible to significantly increase the heating power of the Peltier elements 22 in a “boost” mode for a certain period of time, for example at the beginning of a heating process, in order to shorten the waiting time until the target temperature is reached in the eye pads 1 or the Peltier element surface in the receiving region 23.

Further, the control unit 30 is configured to take into account an ambient temperature measured by an ambient temperature sensor in the controlled heating or cooling power. The control unit 30 increases, reduces or stops the heating or cooling power in predetermined temperature ranges of the ambient temperature. For example, in an environment where the temperature is higher than 55° C., for example, further heating of the eye pad can be prevented, as in this case a malfunction or incorrect operation can be assumed. Alternatively, or additionally, the control unit 30 is further configured to supply the heating or cooling power of the Peltier element 22 in phases to only one of the two receiving regions 23 if the user wishes to simultaneously both cool the receiving region 23 corresponding to the cold side and heat the receiving region corresponding to the warm side and activates this in each case. However, the present invention is not limited to the embodiment variant with a Peltier element, but can also comprise only some of the features described. Thus, for example, a temperature control device 21 can be provided with two or more Peltier elements 22, which, for example, can also be operated independently of one another.

The control unit 30 preferably also comprises a communication interface, which is configured to produce a data connection with a computer unit. The computer unit can, for example, be configured as a smartphone, server or tablet computer, whereby the control unit 30 is configured to regulate the power of the Peltier element 22 on the basis of data received from the computer unit via the communication interface.

In alternative embodiments, the temperature control device 21 may further comprise, for example, an illumination device which illuminates the eye pads 1 in particular with UV light. The illumination device can optionally be disposed in the cover, on the housing or in another manner such that a large part of the eye pad 1, in particular the skin contact surface, is illuminated by the illumination device. In this way, the number of bacteria/viruses/germs that are present, for example, on the surface of the eye pads 1 can be reduced.

What is claimed is:

1. A temperature control device for face pads, comprising:
a Peltier element having a receiving region for receiving a face pad, wherein the receiving region corresponds to a warm side or a cold side of the Peltier element; and
a retaining magnet apparatus for the magnetic fastening of a face pad in the receiving region, wherein the retaining magnet apparatus is disposed in relation to the Peltier element on the side facing away from the receiving region.

2. The temperature control device of claim 1, wherein the Peltier element at least in the receiving region comprises a coating for the mechanical protection of the Peltier element surface.

3. The temperature control device of claim 2, wherein the coating has a thickness of at most 200 μm, preferably at most 100 μm.

4. The temperature control device of claim 1, further comprising a chargeable energy store which is electrically connected to the Peltier element.

5. The temperature control device of claim 1, wherein the retaining magnet apparatus comprises at least two magnets or a magnetizable material for magnetically fastening a face pad.

6. The temperature control device of claim 1, further comprising a fixing device which fixes the Peltier element on the side of the receiving region.

7. The temperature control device of claim 1, wherein in the receiving region at least one temperature sensor is disposed on a surface of the Peltier element and/or in the Peltier element at least one temperature sensor is embedded, and a control unit is connected to the temperature sensor and the Peltier element, the control unit being configured to regulate a heating or cooling power of the Peltier element on the basis of a temperature of one or more of the Peltier element surfaces detected by the temperature sensor.

8. The temperature control device of claim 7, wherein the control unit is further configured to regulate the heating or cooling power of the Peltier element when in the receiving region a target temperature of the Peltier element surface is reached, such that the temperature of the Peltier element surface remains substantially constant.

9. The temperature control device of claim 7, wherein the control unit is further configured to take into account an ambient temperature measured by an ambient temperature sensor in the controlled heating or cooling power in such a way that the heating or cooling power is increased, reduced or prevented in predetermined temperature ranges of the ambient temperature.

10. The temperature control device of claim 7, comprising at least two Peltier elements being in operation simultaneously.

11. The temperature control device of claim 10, wherein the control unit is further configured to supply the current from a power source in phases to only one of the at least two Peltier elements.

12. The temperature control device of claim 8, wherein the control unit is further configured to take into account an ambient temperature measured by an ambient temperature sensor in the controlled heating or cooling power in such a way that the heating or cooling power is increased, reduced or prevented in predetermined temperature ranges of the ambient temperature.

13. The temperature control device of claim 8, comprising at least two Peltier elements being in operation simultaneously.

14. The temperature control device of claim 13, wherein the control unit is further configured to supply the current from a power source in phases to only one of the at least two Peltier elements.
